(19) United States
(12) Patent Application Publication
(10) Pub. No.: US 2015/0018434 A1
(43) Pub. Date: Jan. 15, 2015

(54) COMPOSITION FOR ORAL DELIVERY OF VETERINARY DRUGS AND SUPPLEMENTS TO ANIMALS

(71) Applicant: Barbara Cappelli, Kealakekua, HI (US)

(72) Inventor: Barbara Cappelli, Kealakekua, HI (US)

(21) Appl. No.: 14/381,105

(22) PCT Filed: Feb. 26, 2013
(86) PCT No.: PCT/US2013/027741

§ 371 Date: Aug. 26, 2014

Related U.S. Application Data
(60) Provisional application No. 61/603,704, filed on Feb. 27, 2012.

(51) Int. Cl. A61K 47/36; A61K 9/06; A23K 1/16; A61K 47/42; A61K 47/44

(52) U.S. Cl. CPC A61K 47/36; A61K 47/42; A61K 47/44; A23K 1/1631; A23K 1/1643; A23K 1/164; A61K 9/06
USPC 514/778; 426/656; 426/657; 426/2

(57) ABSTRACT

Crushed pills containing medicines and/or supplements for animals are mixed with an edible paste formulated from all naturally occurring ingredients including specific starches that when mixed together bind the composition to the crushed pills. The mixture is designed to stick to animal fur as well as been very palatable to animal. The medicine is delivered to the animal by offering the paste from a spoon or finger or by smearing the paste on the animal, where it readily adheres to the fur and may be licked off. To encourage finicky animals such as cats to accept the composition from a spoon or finger, and to make it a pleasurable experience for them, the composition includes a desirable food such as real salmon or real meat.

### CROSS-REFERENCE TO RELATED APPLICATIONS

The present application claims priority to U.S. Provisional Patent Application No. 61/603,704, filed Feb. 27, 2012. The contents of that application are hereby incorporated by reference.

### TECHNICAL FIELD

The invention relates to the administration of veterinary medicines and supplements to animals and, more specifically, to a composition that is mixed with the medicine or supplement to create a paste that adheres to fur while masking the taste of the medicine with a taste that is appealing to the animal.

### BACKGROUND

Providing medicines and medicinal supplements to animals orally is a difficult task. The owner often attempts to place the medicine directly in the animal's mouth, only to have the animal spit out the medication or, worse yet, scratch or bite the owner. To avoid this challenge, animal owners may mix the medicine with the animal's food, only to have the animal eat around the medicine or refuse to eat any of the “tampered with” food. For these and other reasons, it is difficult to administer oral medications to animals such as cats and dogs

Several in the prior art have attempted to address these issues by designing containers and other oral medicine delivery systems for animals. Such containers are used to administer capsules of tablets to domestic animals, such as cats and dogs, by providing a body into which the capsule or tablet is inserted. For example, U.S. Pat. No. 5,792,470 discloses an edible tubular member into which the medicine is inserted, while U.S. Pat. No. 5,895,662 and US 2005/0175685 disclose a hollowed pet treat or capsule into which the medicine is inserted. Other such systems include a hollow shell into which a hairball treatment including a lubricant is inserted (US 2005/0084517) or a cooked edible structure enclosing a pouch into which the medicine is inserted (US 2005/0255148 and DE 20 2005 010658). Unfortunately, such systems have limited effectiveness due to unappealing taste or loss of the medicine once the container is bitten into.

Other approaches that have had some success include orally administering the medicine to the animals in a composition made of nonvolatile oils, silicone dioxide, or triglycerides. For example, U.S. Pat. No. 5,122,377 describes an oral delivery system that delivers the medications to the animals in such compositions along with flavorings, oils, and preservatives as desired. Approximately 60-80% of the final composition is triglycerides, 1-5% is silicon dioxide, and 1-20% is the medicine. The composition may be administered in a paste that is ejected into the mouth of the animal through a tube or syringe or that is applied to the animal's paw to be licked.

Similarly, U.S. Pat. No. 7,923,023 describes a method and composition for delivering single doses of medicines to animals, such as bovines, by applying a composition containing a vaccine or pharmaceutical to the muzzle area of the animal for licking by the animal. The composition may have a composition with a viscosity and concentration suitable for application by a brush or roller, as a liquid spray (with or without atomization), or as a paste, salve, or film (such as a tape, adhesive strip, or patch). Ingredients may be added to enhance the aroma of the composition to make it more appealing to the animal, and preservatives are used to maintain the composition for a period of time.

In yet another approach, a composition designed to encase or encapsulate a solid form medication such as a pill is described in US 2006/0246116. The composition surrounding the medication is designed to trigger a dog's gulp reflex. However, dogs have the ability to smell and to identify each ingredient in foodstuffs and thus often find the medication, which is often bitter, and either refuse to accept the composition or refuse the composition the next time it is offered. On the other hand, when applied to cats, the composition is unlikely to be accepted even once. Since cats do not have a gulp reflex, cats taste the medication and either spit it out or refuse it the next time it is offered. Moreover, since the pill is encapsulated in the composition, once the dog or cat bites into the composition, they bite into the pill that is located inside the molded compound, thereby risking that a powdery medicinal substance may stick to the back of the throat of the dog or cat and irritate the esophagus. It is noted that the pill may be crushed to granulate the medication or supplement prior to being encapsulated; however, this still leaves a powdery substance that could potentially stick to the back of the animal's throat and cause irritation.

A new method and composition for delivering medications and/or supplements to animals, such as cats and dogs, is desired that is easy to use, better tasting, and less harmful to the animal. It is also desired to provide a delivery composition that is all natural without non-naturally occurring chemicals and preservatives that may harm the animal. The present invention addresses these needs in the art.

### SUMMARY

The invention addresses the above-mentioned needs in the art by providing a way to safely and easily deliver pills to animals such as cats or dogs. In accordance with the methods of the invention, crushed pills containing the medicines and/or supplements are mixed with an edible paste formulated from all naturally occurring ingredients using specific starches that when mixed together bind the composition to the crushed pills. The mixture can then be delivered in either of two ways: 1) it can be offered from a spoon or finger, or 2) smeared on the animal, where it readily adheres to the fur and will be licked off.

To encourage finicky animals such as cats to accept the composition from a spoon or finger, and to make it a pleasurable experience for them, the composition includes a desirable food such as real salmon. The taste has been found to be so appealing to many animals that it is not always necessary to put it on their fur.

However, it is a known fact that cats will not accept the same foods day after day, even ones they love. So, if a cat rejects the composition from a spoon or finger, it can then be put on the animal's fur. Since cats are so fastidious, they have no choice but to lick it off, thereby ingesting both paste and pill. Such a delivery system provides a calm and safe atmosphere, avoiding any struggle or injury to either human or animal. It can be applied to the fur when the animal is asleep.

The composition is made with only the most nutritious and wholesome naturally occurring ingredients, without non-naturally occurring chemicals or preservatives, and as such has no side effects and can be given on a daily basis. However, the composition is contraindicated for use with enteric or time-release pills.

### DETAILED DESCRIPTION OF ILLUSTRATIVE EMBODIMENTS

Those skilled in the art will appreciate that the description given herein is for exemplary purposes only and is not intended in any way to limit the scope of the invention. All questions regarding the scope of the invention may be resolved by referring to the appended claims.

In accordance with the delivery method of the invention, a medicine or supplement in pill form is placed inside of a small amount of paste and allowed to soften for a few minutes before mixing with the delivery composition. It can also be crushed into small pieces or a powder for mixing with a delivery composition. In an exemplary embodiment, the delivery composition is made of all naturally occurring ingredients that are desirable to the animal. No non-naturally occurring chemicals or preservatives are used. For example, a delivery composition designed for a cat may include real salmon (e.g., pink Alaskan salmon), water, corn starches, salt, natural aroma (e.g., natural smoke flavor), The corn starch functions as a natural binder and thickener that forms the composition into a paste. The corn starch also functions as a food stabilizer and a flavor release enhancer. Delivery compositions for other animals may include other meats and/or flavors known to be desirable to the animal. Alternatively, the delivery composition may include canola oil, pregelatinised rice starch (natural binder and thickener that forms a paste and is highly digestible, a food stabilizer, and neutral in taste), and/or an oleoresin in desirable quantities.

### Example:

To create the delivery composition for commercial applications, the above ingredients are mixed in the indicated weight percentages as follows:

50%-59% of cooked smoked Pink Salmon, (55% ideal)

35% -44% Water (43% ideal)

4% -10% Corn Starch (4.5% ideal for each Corn Starch)

Four hundred and fifty three grams of composition is created by mixing the following products in approximately the indicated amounts by weight percent until a paste is formed:

249 grams of cooked smoked salmon

195.2 grams of water

4.50 grams of corn starch Novation 2300

4.50 grams of corn starch Novation 5300

The cooked smoked salmon is pureed in a blender until very smooth. The Corn Starch Novation 2300 is mixed with the water. The Corn Starch Novation 5300 is added to the water mixture, stirred well, and added immediately to the pureed salmon. The mixture is blended until homogeneous. The mixture is poured into tubes, the tubes are sealed, and retorted (distill, sterilize or decompose by heat in a vessel or chamber) to make the composition shelf stable.

The Corn Starch Novation 5300 is a cold water swelling (CWS) functional native waxy maize food starch that is readily dispersible that has properties similar to modified CWS starches while meeting the labeling criteria of native starches. Corn Starch Novation 5300 provides bright flavor and is suited to withstand processing intensity. The Corn Starch Novation 2300, on the other hand, is a waxy maize functional native starch ideally suited for high temperature/high shear food processing or low pH systems that also has properties similar to modified CWS starches while meeting the labeling criteria of native starches. Corn Starch Novation 2300 adds texture and stability to food products that undergo the heat and shear extremes of food processing. Those skilled in the art will appreciate that the Corn Starch Novation 5300 will “disappear” during the retort process while the Corn Starch Novation 2300 will become activated during the retort process. Those skilled in the art will appreciate that substituting two rice starches for these corn starches will yield different results. No refrigeration is needed until a container of the product is opened.

The crushed pill is mixed with the delivery composition and used to administer daily doses of prescribed medication or supplements by providing the composition orally to the animal by spoon or finger or by placing on the animal's fur to be licked off

The resulting paste is not intended to conceal a solid form medication but instead to blend with the medication or supplement in such a fashion that the animal is unable to distinguish between the paste and the medicine or supplement and has a taste selected to be highly desirable to the animal Also, the resulting paste cannot be separated from the medication or supplement once combined, thereby assuring delivery of the full dosage of the prescribed medication or supplement. The resulting paste is also designed to stick to animal fur as well as to be very palatable to the animal.

Those skilled in the art will also appreciate that the invention may be modified without departing from the scope of the invention. For example, those skilled in the art will appreciate that other fish, meats, and vegetable protein sources (such as tofu) may be used in place of the salmon to formulate a delivery composition that appeals to different animals. Those skilled in the art also will appreciate that the weight percentages of the ingredients may be adjusted outside of the indicated ranges for different delivery compositions based upon the fat content of the fish or meat used and the amount of corn starch used. Also, the mixtures and types of corn starch used may be varied based upon availability and desired paste consistency as well as other varieties of starches such as rice starch or potatoes starch. Those skilled in the art will further appreciate that in order to integrate vitamins such as fish oils into the delivery paste that the starch amount may need to be increased to, for example, 20%, and that water may need to be substituted with fish oils and/or canola omega 3 oil as in the case where Omega 3 additives are added to the delivery paste. The compounding of the paste and the packaging should be done under nitrogen to minimize the oxygen present in the mixture. Exposure to heat should be limited to the minimum amount possible since every minute counts. However, those skilled in the art will appreciate that the composition is contraindicated for use with enteric or time-release pills. Accordingly, the scope of the invention is not intended to be limited to the exemplary embodiments described above, but only by the appended claims.

What is claimed:

1. A composition for delivering medicine or supplements to animals, comprising ingredients including protein, starch, and the medicine or supplement in crushed pill form, each of said ingredients being mixed together into a paste.

2. The composition of claim 1, wherein the protein comprises meat, fish, and/or vegetable protein.

3. The composition of claim 1, wherein the starch is a corn starch, a rice starch, or a potatoes starch.

4. The composition of claim 1, further comprising salt and water.

5. The composition of claim 1, further compromising a natural aroma.

6. The composition of claim 1, wherein the ingredients include by weight percentages approximately 50-59% protein, 35-45% water, and 4-10% corn starch.

7. The composition of claim 1, further comprising fish oil and/or canola omega 3 oil.

8. A method of delivering a medicine or supplement to an animal, comprising the steps of:
creating a paste comprising mixed ingredients including protein, starch, and the medicine or supplement in crushed pill form; and
providing the paste orally to the animal by spoon or finger or by placing the paste on the animal's fur to be licked off by the animal.

9. A method as in claim 8, wherein the step of creating the paste comprises the steps of:
creating a starch mixture by combining corn starch with water;
creating a protein mixture by pureeing the protein;
adding the starch mixture to the pureed protein and blending until homogeneous to create a delivery composition; and
mixing the crushed pill with the delivery composition.

10. A method as in claim 9, comprising the further step of retorting the paste until said paste is shelf stable.

11. A method as in claim 9, wherein the paste ingredients include by weight percentages approximately 50-59% protein, 35-45% water, and 4-10% corn starch.

12. A method as in claim 8, wherein no non-naturally occurring chemicals or preservatives are added to the paste prior to providing the paste to the animal.
